(19) United States
(12) Patent Application Publication
(10) Pub. No.: US 2022/0240793 A1
(43) Pub. Date: Aug. 4, 2022

(54) DETECTION SYSTEM, DETECTION METHOD AND SENSING DEVICE FOR DETECTING STENOSIS OF CAROTID ARTERY

(71) Applicant: Above Care Inc., San Jose, CA (US)

(72) Inventor: Yung-Hsin Chen, San Jose, CA (US)

(73) Assignee: Above Care Inc., San Jose, CA (US)

(21) Appl. No.: 17/719,397

(22) Filed: Apr. 13, 2022

Related U.S. Application Data
(63) Continuation-in-part of application No. 16/388,888, filed on Apr. 19, 2019.
(60) Provisional application No. 62/660,944, filed on Apr. 21, 2018.

(51) Int. Cl. A61B 5/02; A61B 5/00; A61B 7/00

(52) U.S. Cl. CPC A61B 5/02007; A61B 5/0022; A61B 7/00; A61B 5/7267

(57) ABSTRACT

A detection system and a detection method and a sensing device for detecting stenosis of carotid artery are provided. The invention detection system for detecting a stenosis of a carotid artery includes a sensing device and a server. The sensing device includes a microphone. The microphone receives a frequency spectrum signal from a first location. There is a first distance between the first location and a second location of at least one of a plaque and a thrombus in the carotid artery. The first location is located on an extended path of the carotid artery. The server receives the frequency spectrum signal and calculates a stenosis percentage of the carotid artery corresponding to the frequency spectrum signal through a machine learning module and transmits the stenosis percentage to the sensing device.

### CROSS-REFERENCE TO RELATED APPLICATION

This application is a continuation-in-part application of and claims the priority benefit of U.S. application Ser. No. 16/388,888, filed on Apr. 19, 2019, now pending, which claims the priority benefit of U.S. provisional application No. 62/660,944, filed on Apr. 21, 2018. The entirety of each of the above-mentioned patent applications is hereby incorporated by reference herein and made a part of this specification.

### BACKGROUND

### Technical Field

The invention relates to a detection system, a detection method and a sensing device for detecting a carotid artery, and particularly relates to a detection system, a detection method and a sensing device for detecting a stenosis percentage of the carotid artery.

### Description of Related Art

A stenosis of a carotid artery is an abnormal narrowed state of a carotid artery passage, which is a main cause of carotid artery dysfunction. In the current practice, doctors evaluate a condition of the carotid artery irregularly, mostly none. However, the carotid artery stenosis plays the major part of causing the ischemic stroke, which accounts for 800,000 first time stroke in the United States alone, nearly 75% later leading to death. Therefore, to quickly and conveniently evaluate the stenosis condition of the carotid artery is a goal of those skilled in the art in this field.

### SUMMARY

The invention is directed to a detection system and a detection method and a sensing device, which are adapted to quickly and conveniently evaluate a carotid artery stenosis.

The invention detection system for detecting a stenosis of a carotid artery includes a sensing device and a server. The sensing device includes a microphone. The microphone receives a frequency spectrum signal from a first location. There is a first distance between the first location and a second location of at least one of a plaque and a thrombus in the carotid artery. The first location is located on an extended path of the carotid artery. The first distance is greater than 0. The server communicates with the sensing device. The server receives the frequency spectrum signal and calculates a stenosis percentage of the carotid artery corresponding to the frequency spectrum signal through a machine learning module and transmits the stenosis percentage to the sensing device.

The invention provides a detection method for detecting carotid arteries stenosis. The detection method includes: using the sensing device to receive a frequency spectrum signal from a first location through a microphone and transmitting the frequency spectrum signal to a server, wherein there is a first distance between the first location and a second location of at least one of a plaque and a thrombus in the carotid artery, and the first location is located on an extended path of the carotid artery, wherein the first distance is greater than 0; and using the server to calculate a stenosis percentage of the carotid artery corresponding to the frequency spectrum signal through a machine learning module and transmitting the stenosis percentage to the sensing device.

The invention provides a sensing device for communicating with a server. The sensing device includes a microphone. The microphone receives a frequency spectrum signal from a first location. There is a first distance between the first location and a second location of at least one of a plaque and a thrombus in the carotid artery. The first location is located on an extended path of the carotid artery. The first distance is greater than 0. The server communicates with the sensing device. The server receives the frequency spectrum signal and calculates a stenosis percentage of the carotid artery corresponding to the frequency spectrum signal through a machine learning module and transmits the stenosis percentage to the sensing device.

Based on the above description, in the detection system, the sensing device contacts any location on the extended path of the carotid artery to receive the frequency spectrum signal and transmits the frequency spectrum signal to the server. The server calculates the stenosis percentage of the carotid artery corresponding to the frequency spectrum signal and transmits the stenosis percentage to the sensing device. The machine learning module further performs a training operation according to a frequency spectrum signal obtained when the sensing device contacts a different location of the patient body, and calculates the stenosis percentage of the carotid artery according to a result of the training operation.

To make the aforementioned more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a block diagram of a detection system according to an embodiment of the invention.

FIG. 2A is a schematic diagram of an artery and a vein before surgery according to an embodiment of the invention.

FIG. 2B is a schematic diagram of the artery, the vein and an AVF after the surgery according to an embodiment of the invention.

FIG. 3A is a schematic diagram of a sensing device according to an embodiment of the invention.

FIG. 3B is a block diagram of the sensing device according to an embodiment of the invention.

FIG. 4A is a frequency spectrum signal of an AVF without a stenosis condition according to an embodiment of the invention.

FIG. 4B is a frequency spectrum signal of an AVF with a severe stenosis condition according to an embodiment of the invention.

FIG. 5A is a schematic diagram of frequency spectrum energy of an AVF after surgery according to an embodiment of the invention.

FIG. 5B is a schematic diagram of frequency spectrum energy of an AVF before surgery according to an embodiment of the invention.

FIG. 6 is a flowchart of machine learning according to an embodiment of the invention.

FIG. 7 is a schematic diagram of a carotid artery according to an embodiment of the invention.

FIG. 8 is a flowchart of a detection method for detecting a stenosis of a carotid artery according to an embodiment of the invention.

FIG. 9 is a flowchart of a detection method for detecting a stenosis of an AVF according to an embodiment of the invention.

### DESCRIPTION OF THE EMBODIMENTS

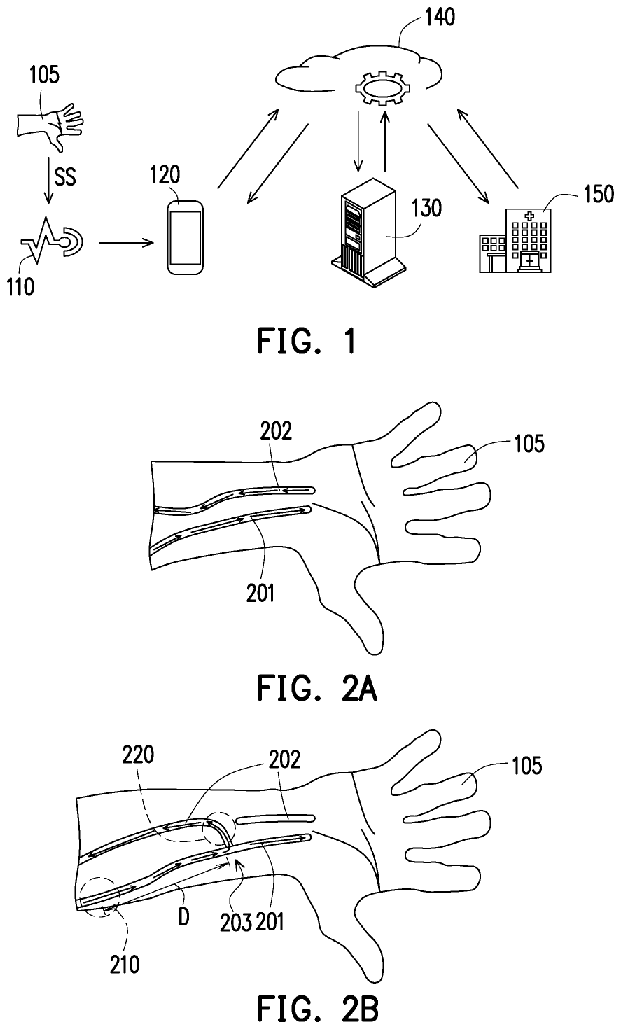

FIG. 1 is a block diagram of a detection system according to an embodiment of the invention.

Referring to FIG. 1, the detection system 100 of an embodiment of the invention includes a sensing device 110, a mobile device 120 and a server 130. The sensing device 110 obtains physiological data from a patient body 105 and transmits the physiological data to the mobile device 120 by using a wireless communication protocol such as WiFi or Bluetooth, etc. The mobile device 120 transmits the physiological data to the server 130 through a wireless network 140 by using a wireless communication protocol such as 4G or 5G, etc. The server 130 may analyze the physiological data through a machine learning module, and transmit back an analysis result to the mobile device 120 to display. Moreover, the server 130 may further transmit the analysis result to a medical organization 150 through the wireless network 140 for patient condition management.

In the embodiment, although a situation that the sensing device 110 is communicated with the server 130 through the mobile device 120 is described, the invention is not limited thereto. In another embodiment, a plurality of sensing devices 110 may also construct an Internet of Things (IOT) network, and may directly communicate with the server 130 through the IOT network. The sensing device 110 may directly transmit the physiological data to the server 130 and obtain the analysis result from the server 130 for displaying on the sensing device 110.

FIG. 2A is a schematic diagram of an artery and a vein before surgery according to an embodiment of the invention. FIG. 2B is a schematic diagram of the artery, the vein and an AVF after the surgery according to an embodiment of the invention.

Referring to FIG. 2A and FIG. 2B, FIG. 2A illustrates an artery 201 and a vein 202 of the patient body 105 before surgery. FIG. 2B illustrates the artery 201, the vein 202 and an AVF 203 connecting the artery 201 and the vein 202 of the patient body 105 after the surgery. In the embodiment, the AVF 203 is located on an arm of the patient body 105, though the invention is not limited thereto. In another embodiment, the AVF 203 may also be located at any part of the patient body 105.

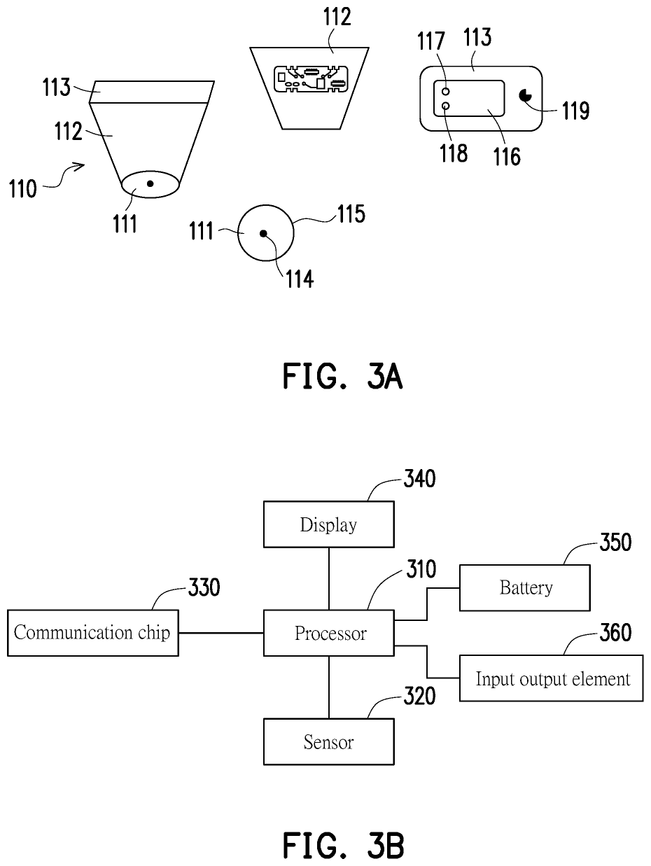

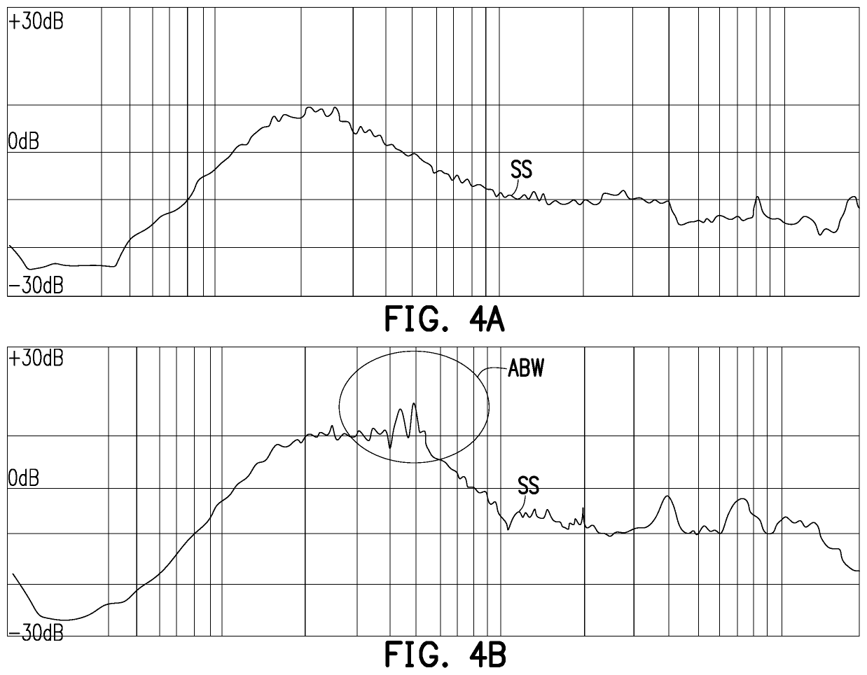

FIG. 3A is a schematic diagram of a sensing device according to an embodiment of the invention. FIG. 3B is a block diagram of the sensing device according to an embodiment of the invention. FIG. 4A is a frequency spectrum signal SS of the AVF without a stenosis condition according to an embodiment of the invention. FIG. 4B is a frequency spectrum signal SS of the AVF with a severe stenosis condition according to an embodiment of the invention.

Referring to FIG. 3A, the sensing device 110 of an embodiment of the invention may include a first portion 111, a second portion 112 and a third portion 113. The first portion 111 may include a microphone 114 and a soft cushion ring 115. Through a construction of the first portion 111, the sensing device 110 may comfortably contact the patient's skin. The second portion 112 may include a circuit structure. The third portion 113 may include a display 116, a power Light-Emitting Diode (LED) 117, a transmission LED 118 and a charging port 119. The display 116 may display an analysis result indicating whether the AVF is stenosing, for example, a stenosis percentage. It should be noted that the microphone 114 may communicates with a confined space of the second portion 112 to reduce a noise generated when the physiological data is obtained. The microphone 114 may include an audio transducer and obtain the physiological data reflected by the patient body 105. Similarly, when the soft cushion ring 115 is closely attached to the patient's skin, the first portion 111 and the patient's skin also form a confined space, so that when the microphone obtains the physiological data, the noise is reduced. The soft cushion ring 115 may include a foamed cotton material without chemical material. The power LED 117 may have different light-emitting modes according to whether the sensing device 110 is in operation or not. The transmission LED 118 may have different light-emitting modes when transmission or non-transmission is carried out. The charging portion 119 is, for example, compatible to at least one Universal Serial Bus (USB) charging specification.

Referring to FIG. 3B, the sensing device 110 of an embodiment of the invention includes a processor 310, a sensor 320 communicates with the processor 310, a communication chip 330, a display 340, a battery 350 and an input output element 360. The sensor 320 is, for example, a sensing head constructed by the microphone 114 and the soft cushion ring 115 of the first portion 111. The sensor 320 may further include a start button (not shown). When the patient presses the start button, the sensor 320 starts to measure the physiological data and automatically stops measuring after, for example, five to ten seconds.

Referring to FIG. 1, FIG. 2B and FIG. 3A, in an embodiment, the sensing device 110 contacts a first location 210 of the patient body 105, where there is a first distance D between the first location 210 and a second location 220 of the AVF 203 of the patient body 105, and the first location 210 is located on an extended path of the artery 201 or the vein 202 corresponding to the AVF 203. The sensing device 110 receives a frequency spectrum signal SS through the microphone 114 and transmits the frequency spectrum signal SS to the server 130. The server 130 calculates a stenosis percentage of the AVF 203 corresponding to the frequency spectrum signal SS through a machine learning module and transmits the stenosis percentage to the sensing device 110. Through the machine learning module, the server 130 may determine that the AVF 203 is in a normal state, for example, the stenosis percentage is equal to 0% through a frequency spectrum signal SS of a normal AVF, for example, the frequency spectrum signal SS of FIG. 4A. On the other hand, the server 130 may also determine that the AVF 203 is in a stenosis state, for example, the stenosis percentage is equal to 80% through a frequency spectrum signal SS of an AVF with a severe stenosis condition, for example, the frequency spectrum signal SS of FIG. 4B. The frequency spectrum signal SS of FIG. 4B includes an abnormal wave form ABW. The abnormal wave form ABW indicates the AVF with a severe stenosis condition.

In an embodiment, when collecting a plurality of frequency spectrum signals corresponding to a plurality of different first locations, the machine learning module performs a training operation by using the frequency spectrum signal SS corresponding to a plurality of different contact locations between the microphone 114 and the patient body 105, and calculates the stenosis percentage according to a result of the training operation. To be specific, the frequency spectrum signal SS is a corresponding sound signal. The sound signal has a resonance characteristic in a blood vessel. Even if the contact location between the microphone 114 and the patient body 105 is not on the AVF 203, as long as the patient places the microphone 114 on the extended path of the artery 201 or the vein 202 corresponding to the AVF 203, the microphone 114 may sense the frequency spectrum signal SS corresponding to the AVF 203. By inputting the frequency spectrum signal SS s corresponding to different locations of the microphones 114 to the machine learning module, a precise prediction result of the stenosis percentage of the AVF 203 may be obtained.

In an embodiment, the server 130 receives angiography information of the patient body 105 and determines a real stenosis percentage according to the angiography information, and the machine learning module trains a plurality of parameters according to the real stenosis percentage to correct the stenosis percentage. In this way, the server 130 may use the exact data of the patient's body 105 to correct the prediction result of the machine learning module, and provide machine learning module to make more accurate prediction of the patient's stenosis percentage.

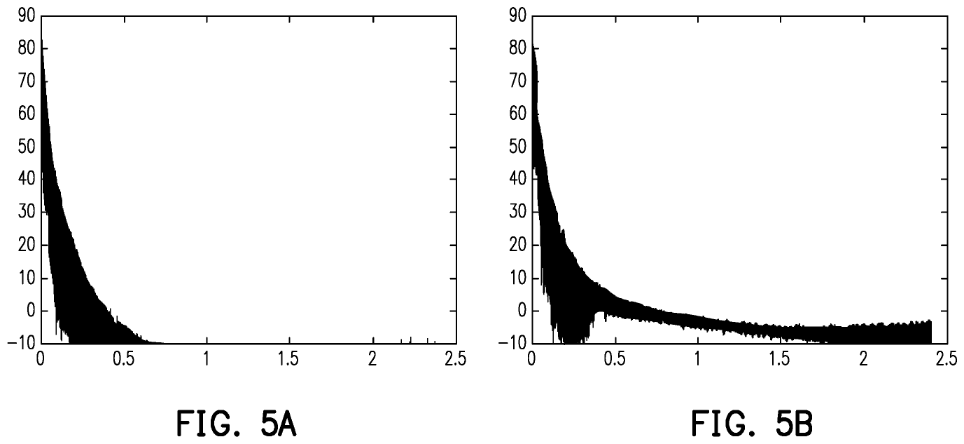

FIG. 5A is a schematic diagram of frequency spectrum energy of the AVF after surgery according to an embodiment of the invention. FIG. 5B is a schematic diagram of frequency spectrum energy of the AVF before surgery according to an embodiment of the invention.

Referring to FIG. 5A and FIG. 5B, in the FIG. 5A and FIG. 5B, a horizontal axis represents frequency and a unit there of is Hertz, and a vertical axis represents energy, and a unit thereof is dB. According to FIG. 5B, it is clearly known that the AVF has high frequency energy before surgery, and according to FIG. 5A, it is obvious that the AVF does not have the high frequency energy after the surgery.

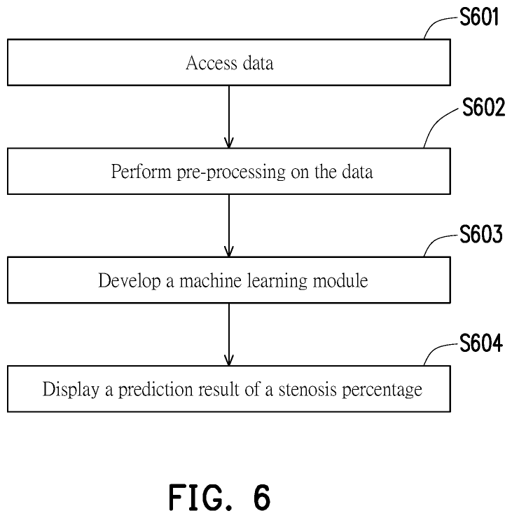

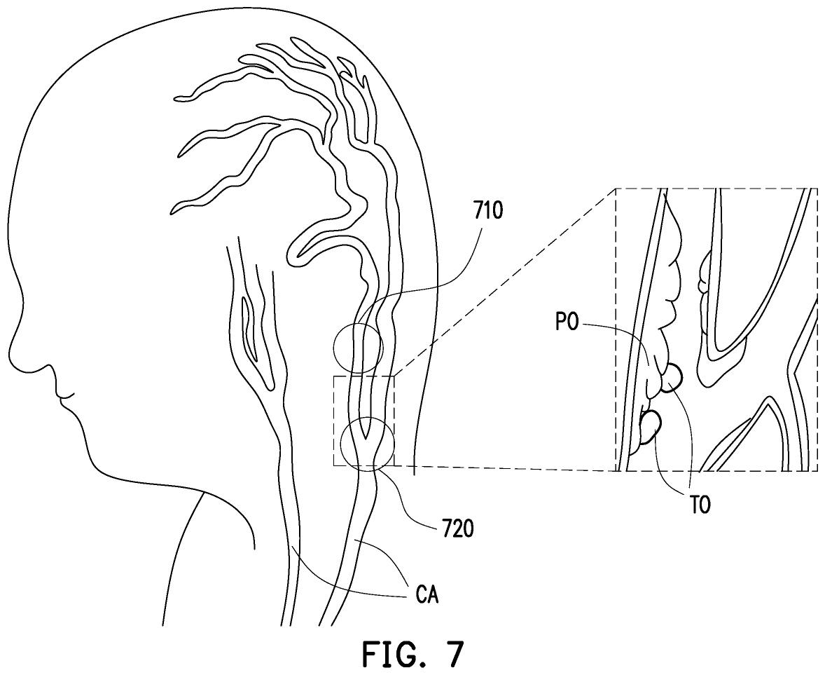

FIG. 6 is a flowchart of machine learning according to an embodiment of the invention.

Referring to FIG. 6, in a step S601, data is accessed. To be specific, the sensing device 110 may obtain the physiological data from the patient body 105, and the physiological data is, for example, a frequency spectrum signal.

In a step S602, the data is pre-processed. To be specific, the server 130 may receive the frequency spectrum data from the sensing device 110, and transform the frequency spectrum data of raw data into a data format suitable for machine learning.

In a step S603, the machine learning module is developed. To be specific, the machine learning module may first obtain a plurality of influencing parameters, for example, at least one of age information, gender information, blood pressure information, the second location, patient historic data, and big data of the server 130. Then, the machine learning module performs feature extraction, parameter optimization, cross comparison, etc., on the format-transformed frequency spectrum data.

In a step S604, a prediction result of a stenosis percentage is displayed. To be specific, after the machine learning module is established, as long as the server 130 receives one batch of frequency spectrum data, the server 130 may generate a corresponding stenosis percentage. The server 130 may send a notification corresponding to the stenosis percentage to display the stenosis percentage on the mobile device 120 and/or the sensing device 110 by ways of email, message or visualization.

Please return to FIG. 1 and FIG. 3B, the detection system 100 and the sensing device 110 are configurated to detect a stenosis percentage of a carotid artery CA.

FIG. 7 is a schematic diagram of a carotid artery according to an embodiment of the invention. Referring to FIG. 1 and FIG. 7. In the embodiment, the sensing device 110 of the detection system 100 contacts a first location 710 of the patient body. There is a distance D between the first location 710 and a second location 720 of at least one of a plaque P0 and a thrombus T0 in the carotid artery CA. The first location 210 is located on an extended path of the carotid artery CA. Besides, the distance D is greater than 0. For example, the distance D is greater than 0 and short than 3 inches. In the embodiment, the first location 710 may be any location of an internal carotid artery. In some embodiments, the first location 210 may be any location of a common carotid artery or an external carotid artery.

In the embodiment, the server 130 communicates with the sensing device 110 and receives the frequency spectrum signal SS from the sensing device 110. The server 130 calculates a stenosis percentage of the carotid artery CA corresponding to the frequency spectrum signal SS through the machine learning module. The server 130 transmits the stenosis percentage to the sensing device 110. In the embodiment, the server 130.

The server 130 calculates a stenosis percentage of the carotid artery CA corresponding to the frequency spectrum signal SS through the machine learning module and transmits the stenosis percentage to the sensing device 110. Through the machine learning module, the server 130 may determine that the carotid artery CA is in a normal state, for example, the stenosis percentage is equal to 0% through a frequency spectrum signal SS of a normal carotid artery CA based on the frequency spectrum signal SS (similar to FIG. 4A). On the other hand, the server 130 may also determine that the carotid artery CA is in a stenosis state, for example, the stenosis percentage is equal to 60% through a frequency spectrum signal SS of an carotid artery CA containing at least one of the plaque P0 and the thrombus T0 based on the frequency spectrum signal SS (similar to FIG. 4B).

It should be noted, FIG. 4A and FIG. 4B is used for detecting a stenosis percentage of the AVF. FIG. 4A and FIG. 4B is also used for detecting a stenosis percentage of the carotid artery CA. However, the detecting frequency domain may be shifted. In other words, A frequency range of the abnormal wave form ABW indicating the AVF with a severe stenosis condition is different from a frequency range of the abnormal wave form ABW indicating the carotid artery CA containing at least one of the plaque P0 and the thrombus T0.

In an embodiment, when collecting a plurality of frequency spectrum signals corresponding to a plurality of different first locations, the machine learning module performs a training operation by using the frequency spectrum signal SS corresponding to a plurality of different contact locations between the microphone 114 and the patient body 105, and calculates the stenosis percentage of the carotid artery CA according to a result of the training operation. To be specific, the frequency spectrum signal SS is a corresponding sound signal. The sound signal has a resonance characteristic in a blood vessel. Even if the contact location between the microphone 114 and the patient body 105 is not on at least one of the plaque P0 and the thrombus T0, as long as the patient places the microphone 114 on the extended path of the carotid artery CA corresponding to the at least one of the plaque P0 and the thrombus T0, the microphone 114 may sense the frequency spectrum signal SS corresponding to the at least one of the plaque P0 and the thrombus T0. By inputting the frequency spectrum signal SS s corresponding to different locations of the microphones 114 to the machine learning module, a precise prediction result of the stenosis percentage of the carotid artery CA may be obtained.

In an embodiment, the server 130 receives angiography information of the patient body 105 and determines a real stenosis percentage of the carotid artery CA according to the angiography information, and the machine learning module trains a plurality of parameters according to the real stenosis percentage to correct the stenosis percentage of the carotid artery CA. In this way, the server 130 may use the exact data of the patient's body 105 to correct the prediction result of the machine learning module, and provide machine learning module to make more accurate prediction of the stenosis percentage of the carotid artery CA.

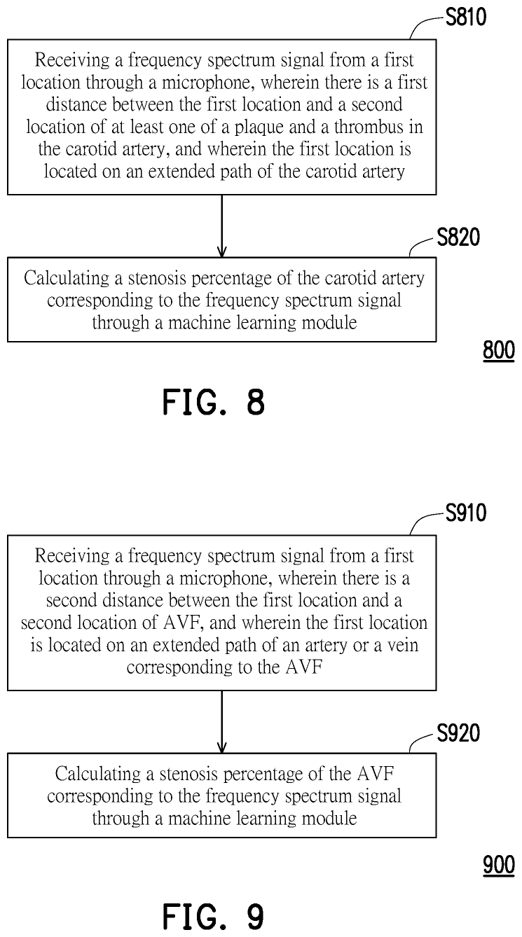

FIG. 8 is a flowchart of a detection method for detecting a stenosis of a carotid artery according to an embodiment of the invention. Referring to FIG. 1, FIG. 7 and FIG. 8, the detection method 5800 includes steps 5810 and 5820. In the step S810, the microphone 114 the frequency spectrum signal receiving the frequency spectrum signal SS from the first location 710. There is a first distance D between the first location 710 and the second location 720 of at least one of the plaque P0 and a thrombus T0 in the carotid artery CA. The first location 710 is located on the extended path of the carotid artery CA. Besides, the frequency spectrum signal SS is transmitted to the server 130 in the step S810.

In the step S820, the server 130 calculates the stenosis percentage of the carotid artery CA corresponding to the frequency spectrum signal SS through the machine learning module. Besides, the stenosis percentage of the carotid artery CA is transmitted to the sensing device 110 in the step S820.

FIG. 9 is a flowchart of a detection method for detecting a stenosis of an AVF according to an embodiment of the invention. Referring to FIG. 1, FIG. 2B and FIG. 9, the detection method S900 includes steps S910 and S920. In the step S910, the microphone 114 receives the frequency spectrum signal SS from the first location 210. There is a first distance D between the first location 210 and the second location 220 of the AVF 203. The first location 210 is located on located on the extended path of the 201 or the vein 202 corresponding to the AVF 203. Besides, the frequency spectrum signal SS is transmitted to the server 130 in the step S910.

In the step S920, the server 130 calculates the stenosis percentage of the AVF 203 corresponding to the frequency spectrum signal through the machine learning module. Besides, the stenosis percentage of the AVF 203 is transmitted to the sensing device 110 in the step S920.

Referring to FIG. 1, FIG. 2B, FIG. 7, FIG.8 and FIG. 9, in the embodiment, the detection system 100 may perform the detection method S800 and the detection method S900 in different modes.

When the sensing device 110 is set in a first mode, the microphone 114 receives the frequency spectrum signal SS from the first location 710 in the step S810. Therefore, in the step S820, the server 130 calculates the stenosis percentage of the carotid artery CA corresponding to the frequency spectrum signal SS from the first location 710 through the machine learning module and transmits the stenosis percentage of the carotid artery CA to the sensing device 110.

When the sensing device 110 is set in a second mode, the microphone 114 receives the frequency spectrum signal SS from a first location 210 in the in the step S910. Therefore, in the step S920, the server 130 calculates the stenosis percentage of the carotid artery CA corresponding to the frequency spectrum signal SS from the first location 710 through the machine learning module and transmits the stenosis percentage of the AVF 203 to the sensing device 110.

In summary, in the detection system, the detection method and the sensing device of the invention, the sensing device contacts any location on the extended path of the artery or the vein corresponding to the AVF to receive the frequency spectrum signal and transmits the frequency spectrum signal to the server. The server calculates the stenosis percentage of the AVF corresponding to the frequency spectrum signal and transmits the stenosis percentage to the sensing device. The machine learning module further performs a training operation according to a frequency spectrum signal obtained when the sensing device contacts a different location of the patient body, and calculates the stenosis percentage of the AVF according to a result of the training operation. Moreover, the server may also receive angiography information of the patient body and determines a real stenosis percentage according to the angiography information, and the machine learning module trains a plurality of parameters according to the real stenosis percentage of the AVF to correct the stenosis percentage.

Besides, the sensing device contacts any location on the extended path of the carotid artery to receive the frequency spectrum signal and transmits the frequency spectrum signal to the server. The server calculates the stenosis percentage of the carotid artery corresponding to the frequency spectrum signal and transmits the stenosis percentage to the sensing device. The machine learning module further performs a training operation according to a frequency spectrum signal obtained when the sensing device contacts a different location of the patient body, and calculates the stenosis percentage of the carotid artery according to a result of the training operation. Moreover, the server may also receive angiography information of the patient body and determines a real stenosis percentage according to the angiography information, and the machine learning module trains a plurality of parameters according to the real stenosis percentage of the carotid artery to correct the stenosis percentage.

It will be apparent to those skilled in the art that various modifications and variations can be made to the disclosed embodiments without departing from the scope or spirit of the invention. In view of the foregoing, it is intended that the invention covers modifications and variations provided they fall within the scope of the following claims and their equivalents.

What is claimed is:

1. A detection system for detecting a stenosis of a carotid artery, comprising:
a sensing device, comprising:
a microphone, configurated to receive a frequency spectrum signal from a first location, wherein there is a first distance between the first location and a second location of at least one of a plaque and a thrombus in the carotid artery, and the first location is located on an extended path of the carotid artery, wherein the first distance is greater than 0; and
a server, configurated to communicate with the sensing device and receive the frequency spectrum signal and calculates a stenosis percentage of the carotid artery corresponding to the frequency spectrum signal through a machine learning module and transmits the stenosis percentage to the sensing device.

2. The detection system as claimed in claim 1, wherein when collecting a plurality of frequency spectrum signals corresponding to a plurality of different first locations, the machine learning module performs a training operation by using the frequency spectrum signal corresponding to plurality of different first locations, and calculates the stenosis percentage according to a result of the training operation.

3. The detection system as claimed in claim 1, wherein the server receives angiography information and determines a real stenosis percentage according to the angiography information, and the machine learning module trains a plurality of parameters according to the real stenosis percentage to correct the stenosis percentage.

4. The detection system as claimed in claim 1, wherein the microphone is coupled to a confined space of the sensing device.

5. The detection system as claimed in claim 1, wherein the machine learning module calculates the stenosis percentage according to a plurality of parameters, and the parameters comprise the frequency spectrum signal, and at least one of age information, gender information, blood pressure information, the second location and patient historic data.

6. A detection method for detecting carotid arteries stenosis, comprising:
using the sensing device to receive a frequency spectrum signal from a first location through a microphone and transmitting the frequency spectrum signal to a server, wherein there is a first distance between the first location and a second location of at least one of a plaque and a thrombus in the carotid artery, and the first location is located on an extended path of the carotid artery, wherein the first distance is greater than 0; and
using the server to calculate a stenosis percentage of the carotid artery corresponding to the frequency spectrum signal through a machine learning module and transmitting the stenosis percentage to the sensing device.

7. The detection method as claimed in claim 6, wherein when collecting a plurality of frequency spectrum signals corresponding to a plurality of different first locations, the machine learning module performs a training operation by using the frequency spectrum signal corresponding to a plurality of different first locations, and calculates the stenosis percentage according to a result of the training operation.

8. The detection method as claimed in claim 6, wherein the server receives angiography information and determines a real stenosis percentage according to the angiography information, and the machine learning module trains a plurality of parameters according to the real stenosis percentage to correct the stenosis percentage.

9. The detection method as claimed in claim 6, wherein the microphone is coupled to a confined space of the sensing device.

10. The detection method as claimed in claim 6, wherein the machine learning module calculates the stenosis percentage according to a plurality of parameters, and the parameters comprise the frequency spectrum signal, and at least one of age information, gender information, blood pressure information, the second location and patient historic data.

11. A sensing device, coupled to a server, and comprising:
a microphone, configurated to receive a first frequency spectrum signal from a first location, wherein there is a first distance between the first location and a second location of at least one of a plaque and a thrombus in the carotid artery, and the first location is located on an extended path of the carotid artery, wherein there is a second distance between the second location and a forth location of an arteriovenous fistula, and the first location is located on an extended path of an artery or a vein corresponding to the arteriovenous fistula, wherein the first distance is greater than 0,
wherein the server calculates a stenosis percentage of the carotid artery corresponding to the first frequency spectrum signal through a machine learning module, and transmits the stenosis percentage to the sensing device.

12. The sensing device as claimed in claim 11, wherein the microphone further receives a second frequency spectrum signal from a third location, wherein there is a second distance between the third location and a fourth location of an arteriovenous fistula, and the third location is located on an extended path of an artery or a vein corresponding to the arteriovenous fistula, wherein the second distance is greater than 0.

13. The sensing device as claimed in claim 12, wherein the server further calculates a stenosis percentage of the arteriovenous fistula corresponding to the second frequency spectrum signal through the machine learning module, and transmits the stenosis percentage to the sensing device.
